(19) United States
(12) Patent Application Publication
(10) Pub. No.: US 2016/0193106 A1
(43) Pub. Date: Jul. 7, 2016

(54) TISSUE REPAIR DEVICES UTILIZING SELF-ASSEMBLED MATERIALS

(71) Applicant: WAKE FOREST UNIVERSITY HEALTH SCIENCES, Winston-salem, NC (US)

(72) Inventors: William D Wagner, Clemmons, NC (US); Nicole Levi, Winston-Salem, NC (US); Rui Wang, Winston-Salem, NC (US); Louis C Argenta, Winston-Salem, NC (US); Michael J Morykwas, Winston-Salem, NC (US)

(21) Appl. No.: 14/911,906

(22) PCT Filed: Aug. 20, 2013
(86) PCT No.: PCT/US2013/055730

§ 371 Date: Feb. 12, 2016

(51) Int. Cl. A61H 9/00; A61L 31/04; A61L 31/06; A61L 31/14

(52) U.S. Cl. CPC A61H 9/0057; A61L 31/146; A61L 31/042; A61L 31/047; A61L 31/06; A61L 2430/20; A61L 2420/06; A61L 2420/02; A61L 2300/606; A61L 2300/252; A61L 2300/236

(57) ABSTRACT

Porous self-assembling biomaterials comprising silk-fibroin and hyaluronic acid and methods for preparing such biomaterials are disclosed. Devices employing the porous self-assembling biomaterials are also provided.

### FIELD OF THE INVENTION

The present invention relates generally to porous self-assembling hydrogels for use as biomaterials and more particularly, but not exclusively, to devices incorporating porous cryogels comprising silk fibroin and hyaluronic acid.

### BACKGROUND OF THE INVENTION

Biomaterials with tissue repair properties are of interest in surgical applications, especially at the device-tissue interface. For example, some current materials may injure tissue upon device removal after concluding treatment and/or may disintegrate at the tissue site leaving pieces of the material behind. These pieces may require removal. Therefore, biomaterials and devices are needed that: 1) allow the function and structure of a device for treating tissue to be retained or improved when positioned at the device-tissue interface; 2) enhance the ease of removal of the device from the tissue site while reducing tissue injury during removal of the device; and 3) promote repair during the use of the device and after the device is removed.

### SUMMARY OF THE INVENTION

In one of its aspects, the present invention answers the needs present in the field by providing methods, materials and devices that include a tissue protective material configured to be placed at the device-tissue interface for use in tissue repair devices.

For example, the invention may provide a sub-atmospheric pressure apparatus for treating a damaged tissue having a nonabsorbable material configured to be placed between a cover and the tissue to be treated. The apparatus may also include a tissue protective material configured to prevent disintegration of the nonabsorbable material and positioned proximate the nonabsorbable material. The protective material may also be configured to be disposed between the nonabsorbable material and the tissue to be treated. The protective material may comprise a porous bioabsorbable cryogel comprising a mixture of silk fibroin and hyaluronic acid. Moreover, the protective material and the nonabsorbable material may be in gaseous communication with each other to allow for the distribution of sub-atmospheric pressure to the tissue to be treated. The protective material may also be injectable. The mixture of silk fibroin and hyaluronic acid may comprise a ratio of silk fibroin to hyaluronic acid of at least about 1.5:1 to 10:1 or at least about 1.5:1, or preferably at least about 2:1. The protective material may comprise a pore size of at least about 15 to 20 μm and may include fenestrations.

The nonabsorbable material may be porous, and may comprise a foam or sponge. The nonabsorbable material may also comprise a synthetic polymer and, more particularly, the synthetic polymer may comprise polyurethane and may include fenestrations. The protective layer may further comprise silicone. Additionally, the apparatus may comprise a source of suction in gaseous communication with the nonabsorbable material and the protective material.

In terms of the positioning of certain elements of the invention, the nonabsorbable material and the protective material may be related in several ways. For example, the protective material may be positioned adjacent to the nonabsorbable material. The protective material may also be located at a selected surface of the nonabsorbable material. Moreover, the protective material may be in contact with the nonabsorbable material such that a portion of the protective material is interspersed or interdigitated within the nonabsorbable material. Indeed, in certain aspects of the invention, and due to the chemical and structural natures of both the nonabsorbable material and the protective material, portions of the protective material may extend into the nonabsorbable material thereby intermixing the protective material with the nonabsorbable material. Such interactions between the nonabsorbable material and protective material may enhance the non-disintegration properties of the invention.

Regarding the devices and methods of the present invention more generally, the application of sub-atmospheric pressure therapy to tissues may provide an increased rate of healing compared to traditional methods (as set forth in U.S. Pat. Nos. 5,645,081; 5,636,643; 7,198,046; 7,216,651; 8,267,960; and 8,377,016, as well as U.S. Published Application Nos. 2003/0225347, 2004/0039391, 2004/0122434, 2009/0187259, and 2010/0121229, the contents of which are incorporated herein by reference).

In another of its aspects, the invention may include a method for preparing a tissue repair material comprising a tissue protective cryogel and a nonabsorbable material. The method may include the step of forming a solution of hyaluronic acid and the silk fibroin. The method may also include the step of applying the solution of hyaluronic acid and the silk fibroin as the tissue protective cryogel to the nonabsorbable material to obtain the tissue repair material.

In one embodiment of the method, the step of applying the solution of hyaluronic acid and silk fibroin as the tissue protective cryogel may comprise lyophilizing the solution of hyaluronic acid and silk fibroin onto a surface of the nonabsorbable material. In another embodiment, the method may comprise the step of preparing silk fibroin by extracting and purifying silk fibroin from raw silk. Moreover, the hyaluronic acid concentration in the solution of hyaluronic acid and silk fibroin may be at least about 30 to 40% by weight. The methods of the invention may also include sonicating and/or vortexing steps.

In at least one of its aspects, the present invention provides biomaterials, and devices the utilize those biomaterials, that may: 1) allow the function and structure of a device for treating tissue and/or damaged tissue to be retained or improved when positioned at the device-tissue interface; 2) enhance the ease of removal of the device from the tissue repair site while reducing tissue injury during removal of the device; and/or 3) promote repair during the use of the device and after the device is removed.

### BRIEF DESCRIPTION OF THE DRAWINGS

The foregoing summary and the following detailed description of the exemplary embodiments of the present invention may be further understood when read in conjunction with the appended drawings (wherein like elements are numbered alike throughout), in which:

FIG. 1 graphically displays three FTIR spectra characterizing (A) an SF-HA cryogel; (B) hyaluronic acid (HA); and (C) silk fibroin (SF).

FIG. 2 graphically displays an evaluation of the elastic modulus of an SF-HA cryogel. Specifically, the force (nN) is compared to the indentation (μm) left in the SF-HA cryogel. Moreover, the resulting experimental data is evaluated using Hertz model fitting.

FIG. 3 displays a scanning electron micrograph (SEM) of an SF-HA cryogel foam exhibiting a porous structure at 1 μm magnification having pores of 15-20 μm.

FIG. 4 displays scanning electron micrographs (SEM) of SF-HA cryogel foam compared to pure fibroin, with panel (A) showing SF-HA cryogel foam at ×230 magnification; panel (B) showing SF-HA cryogel foam at ×900 magnification; panel (C) showing pure fibroin at ×230 magnification; and panel (D) showing pure fibroin at ×900 magnification.

FIG. 5 schematically illustrates a free flowing system used to test the fluid removal ability of a porous material or sponge (e.g., polyurethane foam) coated with an SF-HA cryogel.

FIGS. 6A-6B graphically demonstrate the results of a free flowing system study of fluid removal in sponges composed of polyurethane (PU) foam (control) or PU foam coated with an SF-HA cryogel (SF-HA Coated).

FIG. 7 schematically illustrates a device for testing the performance of SF-HA coated polyurethane (PU) sponges in an ex-vivo sub-atmospheric pressure wound therapy model on liver tissue.

FIG. 8 illustrates the result of applying sub-atmospheric pressure to liver tissue in an ex-vivo model where an SF-HA cryogel coated PU sponge was compared to a non-coated PU sponge (control) and a PU sponge coated with a silicone film, with panel (A) showing tissue examined after 10 minutes of sub-atmospheric pressure application (at −125 mmHg); and panel (B) showing that upon removal of the sponges after 72 hours of sub-atmospheric pressure application at −125 mmHg.

FIG. 9 schematically illustrates a side elevational view in partial cross-section of an exemplary apparatus of the invention in situ showing treatment of a tissue at the surface of the body, wherein the exemplary apparatus includes a protective material.

FIG. 10 schematically illustrates an top elevational view in partial cross-section of another exemplary apparatus of the invention in situ showing treatment of a tissue inside the body, wherein the exemplary apparatus includes a protective material.

### DETAILED DESCRIPTION OF THE INVENTION

Biomaterials with wound healing properties are of interest in medical applications and, particularly, surgical or tissue healing applications. Indeed, there is a need for such biomaterials at the interface between a medical device and a tissue that is being treated or is awaiting treatment. In one of its aspects, the present invention answers those needs.

The materials of the invention, which preferably include hydrogels, may form porous biocompatible, bioincorporable, and/or bioresorbable layers that may (1) prevent tissue injury at a device-tissue interface, allowing for easy removal of surgical devices; and (2) allow the function and structure of the device to be retained or improved when the device is positioned at the device-tissue interface. As used herein, a “bioabsorbable,” “bioresorbable,” or “bioincorporable” material is a material that may dissolve in the tissue or which may be incorporated or absorbed in the tissue as a substantially indistinguishable component.

The hydrogels of the invention include self-assembled porous materials in the form of foams and sponges. More preferably, the hydrogels of the invention are cryogels. As used herein, a “cryogel,” refers to a hydrogel formed in aqueous solution at temperatures below 0° C. Preferably, the self-assembled porous materials (e.g., hydrogels, cryogels) include silk-fibroin (SF) and hyaluronic acid (HA).

The biomaterials of the invention may also be injectable. Indeed, the biomaterials of the invention might also be used in varying degrees of hydration and may be applied to tissue sites using a syringe or other injection device with or without an attached needle. Various degrees of hydration may allow for maintaining the viscosity needed at the tissue location or the hydrogel. Accordingly, the materials of the invention may include an injectable hydrogel that may be injected locally to supplement previously applied materials or devices of the invention. Such sites of local injection could include, for example, damaged tissue, wounds, or surgical sites.

Regarding the material components of the invention, HA may be used as a base component for a hydrogel composite that may be applied to implantable foams or sponges associated with sub-atmospheric pressure therapy systems. Although HA provides wound healing properties to a wound bed and enhanced tissue repair, HA alone may lack the sufficient structural integrity suited for direct use in conjunction with sub-atmospheric pressure due to the mechanical deformation provided by sub-atmospheric pressure. However, HA may be blended or mixed with additional composite materials. The additional composite materials may include, for example, silk fibroin (SF). SF is a unique protein in that it forms beta sheets. Hydrogels may be prepared from combinations of HA and SF. Moreover, following the combination of SF and HA, the hydrogels of the invention may demonstrate beta sheets of SF that entrap HA within the hydrogel.

Hydrogels and cryogels used in device-tissue interface applications may require a significant amount of HA while maintaining stability and functional integrity. Here, materials having different ratios of HA and SF were developed and tested for their physical properties.

Turning to the hydrogels of the invention, the amount of SF is preferably greater than the amount of HA where the hydrogel comprises a mixture of SF and HA. Moreover, the SF-HA hydrogels of the invention may have an SF:HA ratio of at least about 1.5:1 to 10:1. Depending on the application, the SF-HA hydrogels may preferably have an SF:HA ratio of at least about 1.5:1 to 3:1 or, more preferably, the SF-HA hydrogels may have an SF:HA ratio of at least about 2:1. Indeed, in certain applications, a cryogel having an SF:HA ratio of 2:1 may be optimum for the production of a cryogel coating or foam used with implantable nonabsorbable materials. As used herein, a “nonabsorbable” material is defined as a material that may be biocompatible, but is not bioabsorbable, bioincorporable, or bioresorbable. A nonabsorbable material of the invention may be a foam or sponge. Nonabsorbable materials of the invention may comprise polyurethane.

Certain nonabsorbable materials, inserted at the wound and/or tissue site, may have two limitations. For example, certain nonabsorbable materials may cause stress or injury to the tissue site upon removal of the device and/or may disintegrate leaving pieces of the nonabsorbable material at the tissue site that may require removal prior to the cessation of tissue treatment and/or wound closure.

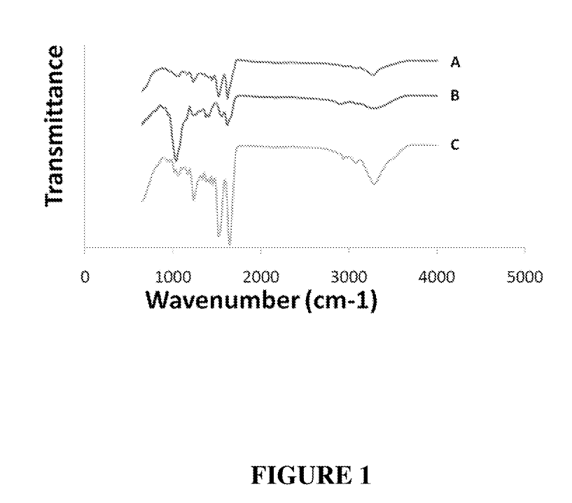

The resulting SF-HA cryogels of the invention were characterized by FTIR and compared to both SF and HA (FIG. 1). The hydrogels of the invention possessed a series of advantageous properties. The hydrogels bind water extensively, possess significant mechanical integrity and stability, and do not impair the fluid removal function of a PU foam after coating the surface of the PU foam with a cryogel of the invention.

The hydrogels of the invention bind water extensively. Indeed, in the hydrogels disclosed herein, the mixture of SF and HA may be proportioned in a ratio that provides a water binding potential of at least 1500% by weight. Enhanced water binding provides a loose aqueous state permitting ease of migration and proliferation of cells in repairing tissue. Preferably, the hydrogels of the invention comprise a water binding potential of at least about 2000% to about 2350%. More preferably, the water binding potential is at least about 2250% to about 2350%. By example, the water binding potential of a composite hydrogel having an SF-HA ratio of 1:1 results in a 15 fold water binding potential (1500%). In comparison, a cryogel of the invention having an SF-HA ratio of about 2:1 provides a water binding potential that is approximately 23 fold (2300%).

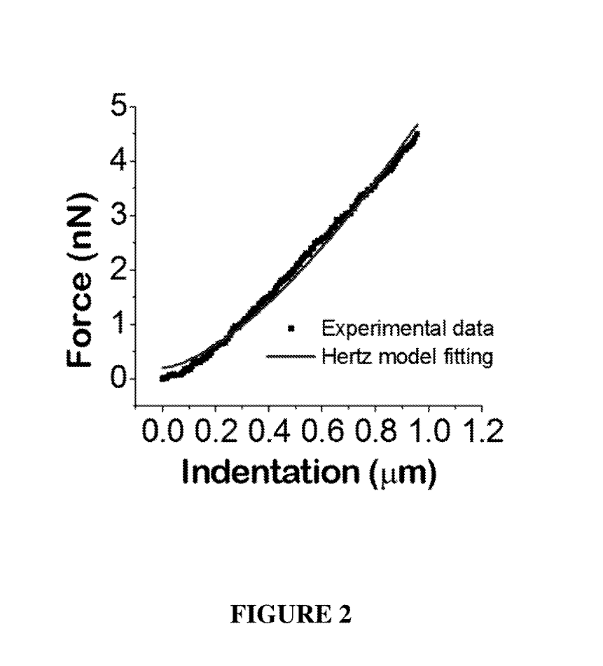

The mechanical properties of the hydrogels of the invention were examined to determine their applicability as relevant biomaterials. The hydrogels disclosed herein possessed significant stability and mechanical integrity as determined by their elastic modulus. Preferably, the hydrogels of the invention have an elastic modulus of at least about 2 to 8 kPa. More preferably, the elastic modulus is at least about 4 to 6 kPa. Most preferably, the elastic modulus is at least about 4.5 to 5.5 kPa. For example, a cryogel of the invention (SF-HA ratio of 2:1) possessed an elastic modulus of 4.97 kPa as determined by Atomic Force Microscopy (AFM) (see FIG. 2).

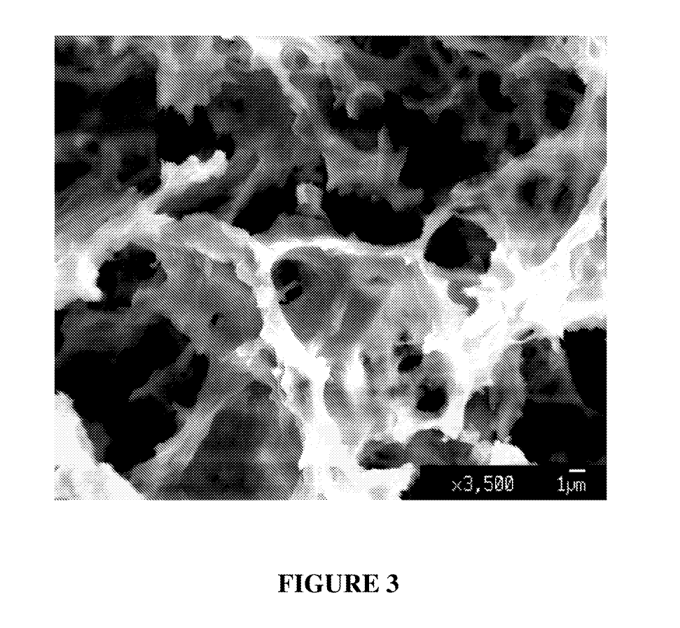

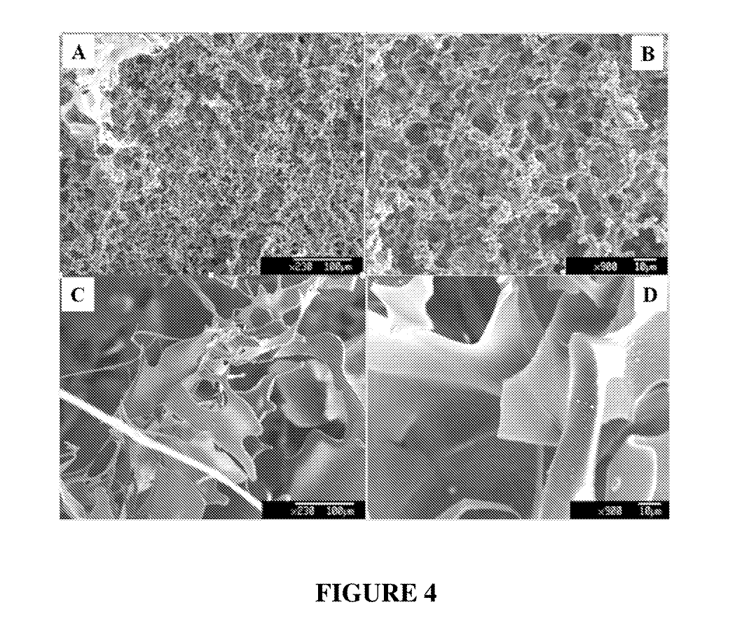

Regarding the structure of the hydrogels of the invention, the hydrogel materials may be porous and have a pore size of at least about 15 to 20 μm. Moreover, the preferred hydrogels of the invention may be self-assembled porous hydrogels. Regarding the aspect of self-assembly, upon gelation the materials of the invention may spontaneously form a porous scaffold or matrix. For example, by scanning electron microscopy (SEM), a cryogel of the invention was found to have a pore size ranging from 15 to 20 μm (FIGS. 3 and 4).

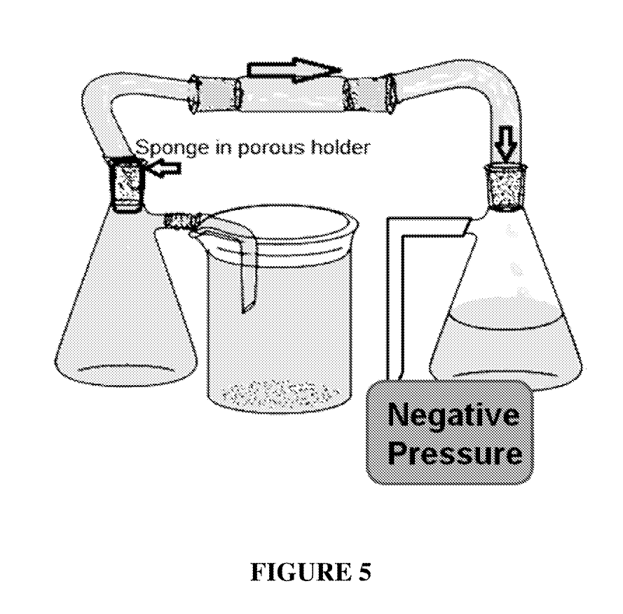

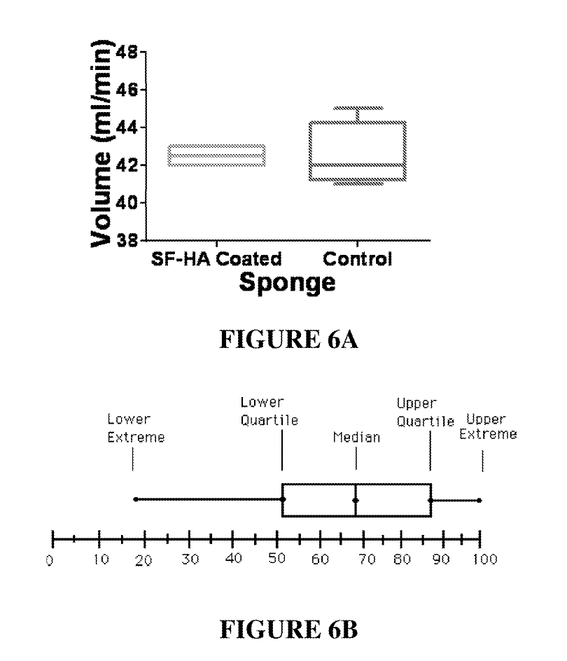

The materials of the invention were also tested after application to PU foam to determine the effect of an SF-HA cryogel coating on the fluid removal function of the PU foam using a free flow system (FIG. 5). This free flow system provides a method of testing fluid removal with a system that is unregulated by filters, cells, and/or tissues. In reference to FIG. 5, the porous material or sponge, coated with an SF-HA cryogel, is placed in a porous holder. Next, fluid is drawn through the coated porous material or sponge by the application of sub-atmospheric pressure downstream from the coated porous material or sponge. By examining the passage of fluid through the coated porous material or sponge the impairment of fluid removal through the coated porous material or sponge, or lack thereof, can be determined. Moreover, FIG. 6A shows a fluid removal graph where the rate of fluid removal was compared between the Control sponge and the SF-HA Coated sponge where the SF-HA coated sponge did not impair fluid removal as compared to the Control sponge and also demonstrated more consistent, less variable fluid flow. FIG. 6B provides a Key for understanding the fluid removal graph in FIG. 6A by noting the graphical location of the Upper Extreme, Upper Quartile, Median, Lower Quartile, and Lower Extreme. Accordingly, fluid removal through PU foam was not impaired by the presence of the cryogel coating as determined using a free flowing system (FIGS. 5, 6A, and 6B). Thus, the fluid in the free flow system has unrestricted flow prior to entering the test foam.

Regarding a preferred preparation of the cryogels of the invention, stable non-aqueous dissolving cryogels having high concentrations of HA were prepared. Generally, SF was obtained after extraction from silk (e.g., using an aqueous solvent processing method). Fibroin was identified based on amino acid analysis, molecular weight and functional group identification. A 0.58% solution of fibroin was made and maintained at 4° C. HA (6 mg) was dissolved in 2 mL of fibroin solution. A homogenous solution was obtained and cryo cast in a cylindrical container with a PU sponge placed on top for cryo casting. The foam composite was placed at −80° C. overnight and then lyophilized for 24 hours to form a cryogel coating on the tissue contacting surface of the PU sponge.

The hydrogel foams of the invention differ from other materials prepared in the field (see, e.g., Hu et al. (2010)). Indeed, in the hydrogel foams of the invention, a homogenous solution of SF and HA produced a stable gel made through a cryogelation process rather than through a primary sonication process that creates energy input and produces molecular interactions in the subject material.

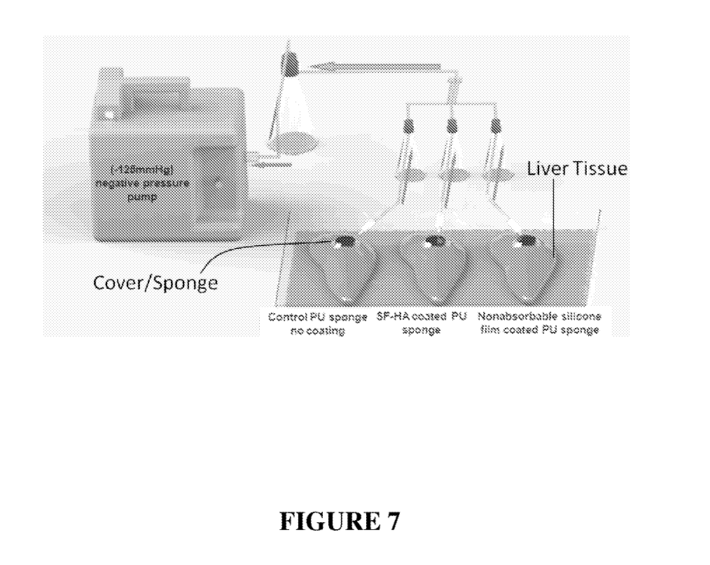

Additionally, an ex-vivo liver model was used to compare the outcomes in a sub-atmospheric pressure device employing 1) a nonabsorbable PU sponge with an SF-HA cryogel layer, 2) the PU sponge without the cryogel layer, and 3) the PU sponge with a silicone film. With reference to FIG. 7, the SF-HA coated PU sponge was compared to a non-coated PU sponge (control) and a PU sponge coated with a silicone film. Specifically, the sponges were placed on liver tissue, then a cover was placed over both the sponge and the tissue to be treated. A sub-atmospheric pressure source was then positioned in gaseous communication with the space under the cover to provide sub-atmospheric pressure through the sponge to the tissue to be treated. Sub-atmospheric pressure was applied at −125 mmHg.

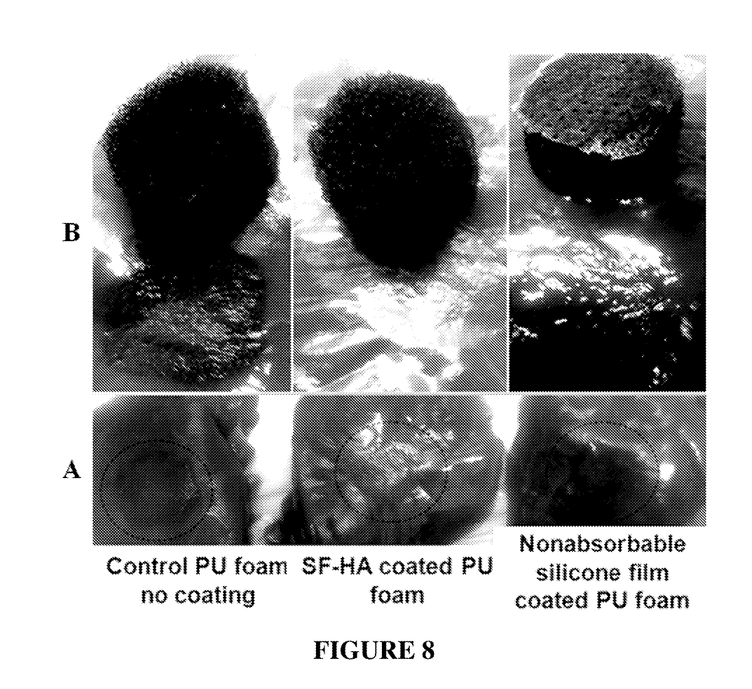

Using the ex-vivo liver model system (FIG. 7) it was demonstrated that in a 72-hour period at −125 mmHg sub-atmospheric pressure, less tissue deformation was observed with the cryogel coated PU sponge as compared to the non-coated or silicone coated PU sponge (FIG. 8). Indeed, as demonstrated in the FIG. 8 images, where sub-atmospheric pressure was distributed to the liver tissue through an SF-HA cryogel coated PU sponge, there was less deformation (stippling at the site of tissue treatment) to the liver tissue observed as compared to the non-coated PU sponge or the sponge coated with a silicone film. In addition, and without being limited to any one theory of operation, it is hypothesized that the blend of SF (strength provider) and HA (tissue repair promoter) optimize tissue repair when used in conjunction with sub-atmospheric pressure therapy.

As demonstrated, SF may be extracted and then fabricated with HA using a cryogelation process. A highly porous and homogenous structure results from the fabrication of SF with HA as compared to SF gel. The SF-HA cryogels of the invention possess water binding ability with an absorption of about 2300±350% (or a 23±3.5 fold weight increase attributed to hydration). The elastic modulus may be about 4.79 kPa. Functional tests on materials of the invention indicate comparable fluid removal of PU foam having an SF-HA coating as compared to a PU foam lacking such coating. Moreover, the nonabsorbable PU foam may be easily removed with the SF-HA protective material resulting in minimal tissue damage when compared to a PU foam lacking such a protective material. Indeed, the invention demonstrates a cryogel made from SF and HA that can be used as a potent tissue protective material in sub-atmospheric pressure treatment therapy to reduce dressing change associated pain and to prevent secondary injury to a wound bed.

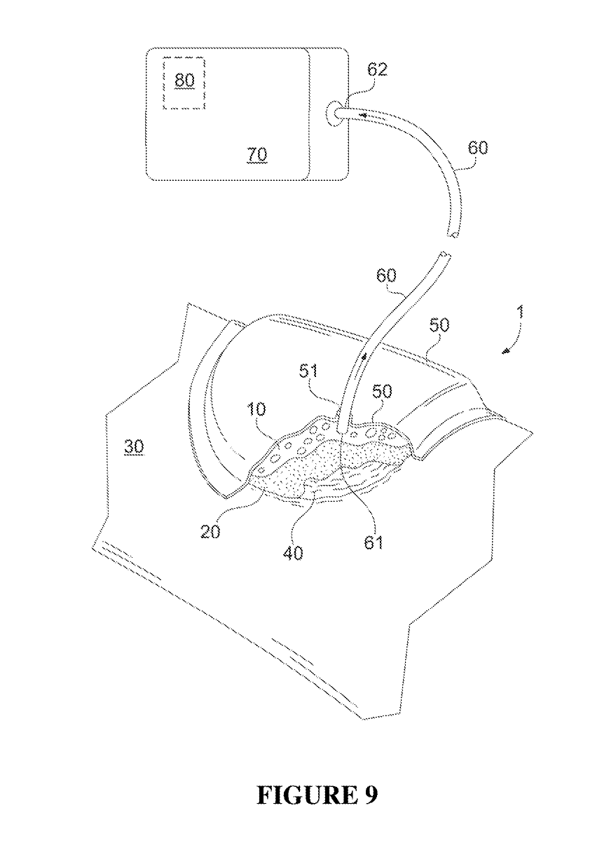

Referring now to FIG. 9, the invention encompasses devices that use sub-atmospheric pressure for treating wounded or damaged tissue, wherein the devices incorporate an SF-HA cryogel as set forth above. As used herein “damaged” tissue is defined to include tissue that is injured, compromised, or in any other way impaired, including damage due to trauma, disease, infection, surgical complication, or other pathological process, for example. An exemplary configuration of a sub-atmospheric treatment device 1 of the invention employing an SF-HA cryogel is portrayed in FIG. 9.

The sub-atmospheric treatment device 1 may deliver and distribute sub-atmospheric pressure to damaged tissue 40. Moreover, treatment device 1 may comprise a porous material 10 disposed proximate the tissue to be treated, such as tissue 40. The porous material 10 may be a nonabsorbable foam or sponge such as a polyurethane (PU) foam or sponge. The porous material 10 may be provided with a tissue protective material 20. The tissue protective material 20 may preferably be an SF-HA cryogel coating or layer that is proximate the porous material 10 and may be disposed between the porous material 10 and the tissue to be treated 40. Regarding additional or alternative positioning modalities of the porous material 10 and protective material 20, the protective material 20 may be positioned adjacent to the porous material 10. The protective material 20 may also be located at a selected surface of the porous material 10, to the exclusion or inclusion of other surfaces of the porous material 10. For example, the protective material 20 may be located at a surface of the porous material selected because such selected surface is proximate to a specific tissue or organ. The protective material 20 may also be in contact with the porous material 10 such that a portion of the protective material 20 is interspersed or interdigitated within the porous material 10.

Indeed, in certain aspects of the invention, and due to the chemical and structural natures of both the porous material and the protective material, portions of the protective material 20 may extend into the porous material 10 thereby intermixing the protective material 20 with the porous material 10. Such interactions between the porous material 10 and protective material 20 may enhance the adhesion of the protective material 20 to the porous material 10, and thus further protect against possible disintegration of the porous material 10.

In usage, the devices of the invention may be deployed to treat tissues and/or organs beneath the surface of the body or damaged tissue on the surface of the body, or may be used in vitro.

Turning to the delivery of sub-atmospheric pressure to the porous material 10 and distribution to the damaged tissue 40 through the porous material 10 and protective material 20, a tube 60 may be connected directly or indirectly in gaseous communication with the porous material 10 at the application end 61 of the tube 60. The application end 61 of the tube 60 may also be embedded in the porous material 10 or it may be placed over the porous material 10. The application end 61 of the tube 60 may also be fenestrated.

A sub-atmospheric pressure source 70 (e.g., a vacuum pump) may be operably connected to the source end 62 of the tube 60. In this fashion, sub-atmospheric pressure may be transmitted via the tube 60 to the porous material 10 and the damaged tissue 40 through the protective material 20.

The sub-atmospheric pressure source 70 may include a controller 80 to regulate the sub-atmospheric pressure application. Indeed, the sub-atmospheric pressure source 70 may be configured to produce sub-atmospheric pressure continuously or intermittently. For example, the sub-atmospheric pressure source 70 may be cycled on and off, thereby providing periods of production and non-production of sub-atmospheric pressure. The operation cycle of the sub-atmospheric pressure may provide varied production and non-production of sub-atmospheric pressure between 1 to 10 (on/off) and 10 to 1 (on/off). Moreover, sub-atmospheric pressure can be applied by a periodic or cyclical waveform (e.g., a sine wave). The sub-atmospheric pressure source 70 may also be cycled after initial treatment to mimic certain physiologic states. For example, the sub-atmospheric pressure may be cycled for several times per minute. Furthermore, the sub-atmospheric pressure may be cycled on-off as needed or as determined by monitoring the pressure in the damaged tissue 40. The sub-atmospheric pressure source 70 may be configured to deliver sub-atmospheric pressure between atmospheric pressure and 125 mmHg below atmospheric pressure (i.e., −125 mmHg).

To assist in maintaining sub-atmospheric pressure at the damaged tissue 40, a cover 50 may be provided proximate the damaged tissue 40 to provide a region of sub-atmospheric pressure maintenance about the damaged tissue 40. The cover 50 may comprise a sheet and/or may be flexible, rigid, semi-rigid, or a combination thereof. Moreover, the cover 50 may also comprise an airtight dressing. Specifically, a cover 50 may be provided over the damaged tissue 40 and porous material 10 having a protective material 20 by adhering the cover 50 to tissues such as skin 30, proximate the damaged tissue 40, to provide an enclosed region about the damaged tissue 40 and porous material 10 having protective material 20. For example, the cover 50 may be glued with an adhesive (e.g., fibrin glue) to the skin 30, other tissues, or a combination thereof. The adhesive may comprise auto-polymerizing glue and/or may desirably include a filler to provide a sufficiently bulky adhesive that permits the adhesive to conform to the regular or irregular surfaces about the treatment site. The adhesive can be provided separately or may be integrated with the cover 50 to provide a self-adhesive cover 50. Indeed, the cover 50 can comprise a flexible self-adhesive sheet that includes a suitable adhesive on one or more of its surfaces.

Sub-atmospheric pressure may be delivered under the cover 50 through cooperation between the cover 50 and the tube 60. Specifically, the cover 50 may include a fixed inlet (not shown) to which the application end 61 of the tube 60 connects to provide gaseous communication between the tube 60 and the space under the cover 50 over the damaged tissue 40 and porous material 10 having protective material 20. The tube 60 may be connected or disconnected from the fixed inlet without breaking the sub-atmospheric pressure maintained under the cover 50. Alternatively, the cover 50 may include a pass-through 51 through which the tube 60 passes so that the application end 61 of the tube 60 is disposed interior to, and in gaseous communication with, the space under the cover 50 over the damaged tissue 40. In addition the cover 50 may further protect the damaged tissue 40 from exogenous infection and contamination beyond the protection already afforded by the porous material 10 and protective material 20. Likewise, the cover 50 may further protect surrounding tissues from the spread of infection from the damaged tissue 40.

In another of its aspects, the invention also provides a method for treating damaged tissue using a sub-atmospheric pressure device or apparatus comprising an SF-HA cryogel coating. In particular, the method may comprise locating a porous material 10, having a protective material 20 (e.g., SF-HA cryogel coating), proximate the damaged tissue 40 to provide gaseous communication between one or more pores of the porous material 10, through the protective material 20, and the damaged tissue 40. The porous material 10 having protective material 20 may be sealed in situ proximate the damaged tissue 40. In this fashion, sub-atmospheric pressure is maintained in a region about the damaged tissue 40. A tube 60 may be connected to the porous material 10 at an application end 61 of the tube 60. A cover 50 may be placed to provide an airtight seal to maintain sub-atmospheric pressure at the tissue. The method may also include adhesively sealing and adhering the cover 50 to tissue (e.g., skin 30) surrounding the damaged tissue 9. The cover 50 may be positioned as a self-adhesive sheet 50 that may be located over the damaged tissue 40. In this manner, sealing the cover 50 may encompass adhesively sealing and adhering the self-adhesive sheet 50 to tissue surrounding the damaged tissue 40. Additionally, operably connecting a sub-atmospheric pressure system 70 in gaseous communication with the porous material 10 may comprise connecting the sub-atmospheric pressure system 70 with a fixed inlet of the cover 50 that allows for the detachability of the sub-atmospheric pressure source 70 without breaching the cover 50.

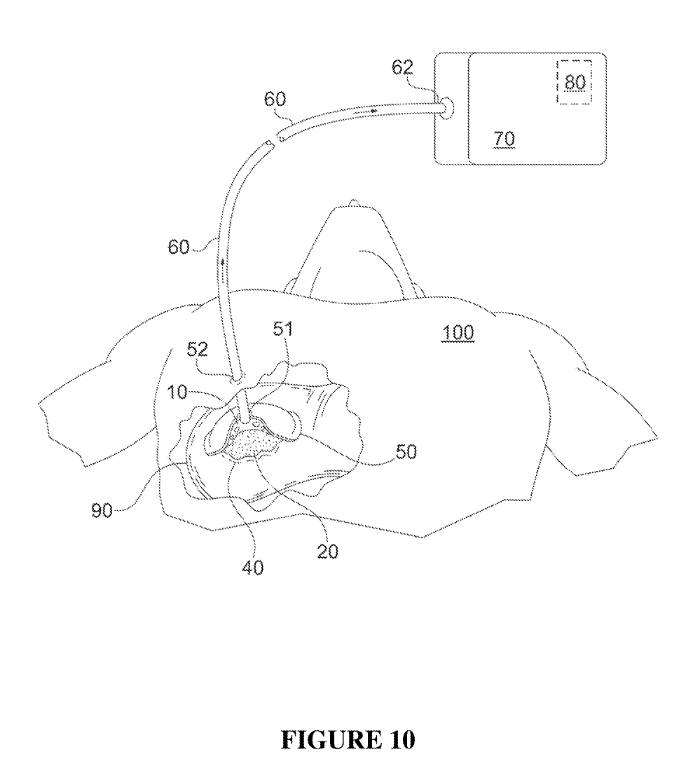

Referring now to FIG. 10, the device of the invention may be placed inside the body 100 to treat damaged tissue 40 and/or an organ 90, such as the liver. In such methods and devices of the invention, the skin and other surface tissues would be opened to expose the damaged tissue 40 and/or organ 90 to be treated. Indeed, the organ 90 may include the damaged tissue 40. The device of the invention could then be placed at the treatment site, e.g., damaged tissue 40 of organ 90, with the device including a porous material 10 having a tissue protective material 20 with a cover 50. A tube 60 may also be placed at the cover 50 through a pass-through 51 to provide gaseous communication between one or more pores of the porous material 10, through the protective material 20, and the damaged tissue 40. Additionally, tube 60 may be configured to pass through the surface of the body 100 via a tissue opening 52. Tissue opening 52 may be an opening in the body that is made specifically for the tube (and cut to the appropriate dimension) or tissue opening 52 may be the opening used to access the damaged tissue 40 and/or organ 90. The tissue opening 52 may be closed around the tube 60 with the aid of sutures, staples, glue, or the like.

In certain configurations, including those exhibited in FIGS. 9 and 10, when the sub-atmospheric pressure source 70 is activated the source end 62 of the tube 60 can be attached to the sub-atmospheric pressure source 70 to apply sub-atmospheric pressure to the damaged tissue 40. For example, the sub-atmospheric pressure may be maintained at about 125 mmHg below atmospheric pressure (−125 mmHg). Alternatively, the sub-atmospheric pressure may vary between atmospheric pressure and 125 mmHg below atmospheric pressure. The sub-atmospheric pressure may be maintained or varied at the damaged tissue 40 for a time sufficient to achieve a selected stage of healing. The method may be used for several hours, or can be used for many days. At the end of vacuum treatment, the porous material 10 may then be removed allowing the protective material 20 to remain if desired. When the tissue to be treated is an organ beneath the skin, the device 1 may be extracted from the body, leaving protective material 20. However, if the tissue to be treated includes a surface wound, the device 1 may be removed, leaving protective material 20, and the skin 30 may then be closed. In alternative configurations, the porous material 10 and protective material 20 may be removed as one unit, or as separate units, prior to skin closure.

The following examples are provided to describe the invention in further detail. These examples are provided for illustrative purposes only and are not intended to limit the invention in any way.

### Example 1

A method for generating the cryogel for coating a polyurethane (PU) sponge useful in a sub-atmospheric pressure treatment system may be prepared as follows:

Silk fibroin (SF) was first extracted and purified from Bombyx mori raw silk. To extract the silk fibroin, sodium carbonate (1060 g) was dissolved in deionized distilled water (2 L) to make a 0.02 M sodium carbonate solution in a beaker. The beaker was covered with aluminum foil and the solution was brought to a boil. Raw silk (10 g) was placed in the boiling sodium carbonate solution for 30 minutes. The residual fibroin silk protein was removed from the solution and squeezed to remove excess water then washed with water. The silk fibroin was rinsed with hot water (approximately 20 mL/gram silk) for 20 minutes. The rinse was repeated three times. The fibroin was then dried at room temperature in a fume hood for 12 hours. The fibroin was stored at room temperature in a clean plastic bag until used. To prepare solutions, silk fibroin was dissolved in 5.0 M CaCl₂ at 100° C. for 3 hours to obtain a clear amber color solution. The solution was removed from heat, cooled and centrifuged at 1000×g for 20 minutes. The resulting supernatant was transferred to a cellulose membrane (Spectrapor: 12,000-14,000 molecular weight cut off) and dialyzed against distilled water exhaustively for 48 hours. The water was changed 6-10 times during dialysis. After dialysis, the silk fibroin solution was transferred into 50 mL conical tubes. The SF solution was stored at 4° C. prior to use. For purity, the final SF was examined after gel electrophoresis, to determine molecular weight, and high performance liquid chromatography (HPLC) to determine amino acid composition. The SF was also tested thermally using a differential scanning calorimeter to determine the T_{g} (T_{g}=178° C.) and Tₘ (Tₘ, =192-203° C.). (See Rockwood, et al. (2011)).

Hyaluronic Acid (HA) (purchased from Sigma Aldrich) was minced into pieces (approximately 1 mm by 1 mm in size) using an iris scissor, weighed, and dissolved in an aqueous solution of SF (0.58%). The final concentration of HA was 34%. The solution remained at 22° C. for 1 hour to dissolve the HA and obtain a homogenous SF-HA solution. Cryogels were obtained following lyophilization for 24 hours with the cryogel having an SF/HA ratio of about 2:1.

When coating a surface of a PU sponge with SF-HA material, SF-HA solution (6 mL) was used and transferred onto a petridish. The PU sponge was placed on top of the SF-HA solution and the entire dish was subjected to lyophilization. The SF-HA and PU sponge was lyophilized for approximately 24 hours until dried to form the assembled cryogel-sponge construct.

### Example 2

Hyaluronic Acid (HA) was minced into pieces (approximately 1 mm by 1 mm in size) using an iris scissor, and dissolved in an aqueous solution of silk fibroin (SF) (0.58%). The solution was sonicated with an ultra-sonicator (Branson Digital Sonifier) 30 seconds for 3 times to dissolve the HA in the silk fibroin (SF) solution. The solution contained excess bubbles. Once the homogenous SF-HA solution was obtained, the solution was transferred to a petridish and lyophilized to form a cryogel. Specifically, PU sponges were coated with SF-HA, the SF-HA solution was placed in the petridish and PU sponge placed on top with the entire dish and material were subject to cryogelation. Some bubbles dissipated over time but this method was not ideal in forming a fully coated surface on a PU sponge.

### Example 3

A method for preparing a material of the invention, and similar to Example 2, applies vortexing to the SF-HA solution rather than ultra-sonication. Indeed, vortexing (Fisher vortex, Genie2, speed8) was used for a period of 30 seconds, and repeated 3 times. The solution contained bubbles. Specifically, PU sponges were coated with SF-HA, the SF-HA solution was placed in the petridish and PU sponge placed on top with the entire dish and material were subject to cryogelation. The method was not ideal in forming a fully coated surface on a PU sponge.

### Example 4

To determine the effect of the SF-HA material of the invention on cells, human umbilical vascular endothelial cells (HUVECs) obtained from ATCC#CRL-1730 were seeded on an SF-HA solution spray-coated glass slide (experimental group) to assess cell viability after 24 hours compared to a polystyrene plastic cell culture plate (control group). The cells were stained with live/death staining. On completion of the study, there was virtually no difference between the experimental group and control group in terms of live/death cell staining.

### Example 5

To determine the effect of temperature on SF-HA cryogels of the invention, an SF-HA cryogel was transferred to a chambered slide at 22° C. (n=7) and 37° C. (n=6) incubator. The glass chambered slide was kept for 24 hours before examining the gel with the unaided eye. There was no difference in the gel morphology between cryogel at 22° C. and 37° C. No liquefaction of the SF-HA cryogels was observed. The cryogel was translucent with no observable changes during the 24 hour time period.

A number of patent and non-patent publications are cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All devices and methods for preparing the same that embody the present invention can, in alternate embodiments, be more specifically defined by any of the transitional terms “comprising,” “consisting essentially of,” and “consisting of”.

### REFERENCES

1. Hu, X., et al. Biomacromolecules 2010, 11 (11), 3178-3188.
2. DeFranzo, A. J. et al., Plastic and Reconstructive Surgery 2001, 108 (5), 1184-1191.
3. Rockwood, D. N., et al., Nat. Protocols 2011, 6 (10), 1612-1631.

1. A sub-atmospheric pressure apparatus for treating a damaged tissue comprising:
a. a nonabsorbable material configured to be placed between a cover and the tissue to be treated; and
b. a tissue protective material configured to prevent disintegration of the nonabsorbable material and positioned proximate the nonabsorbable material, the protective material configured to be disposed between the nonabsorbable material and the tissue to be treated, the protective material comprising a porous bioabsorbable cryogel comprising a mixture of silk fibroin and hyaluronic acid;
wherein the protective material and the nonabsorbable material are in gaseous communication to allow for the distribution of sub-atmospheric pressure to the tissue to be treated.

2. The apparatus of claim 1, wherein the nonabsorbable material is porous.

3. The apparatus of claim 1, wherein the protective material is injectable.

4. The apparatus of claim 1, wherein the nonabsorbable material comprises a foam or sponge.

5. The apparatus of claim 1, wherein the mixture of silk fibroin and hyaluronic acid comprises a ratio of silk fibroin to hyaluronic acid of at least about 1.5:1 to 10:1.

6. The apparatus of claim 1, wherein the mixture of silk fibroin and hyaluronic acid comprises a ratio of silk fibroin to hyaluronic acid is at least about 1.5:1 to 3:1.

7. The apparatus of claim 1, wherein the mixture of silk fibroin and hyaluronic acid comprises a ratio of silk fibroin to hyaluronic acid is at least about 2:1.

8. The apparatus of claim 1, wherein the protective material comprises a water binding potential of at least about 2000% to about 2500%.

9. The apparatus of claim 1, wherein the protective material comprises a water binding potential of at least about 2250% to about 2350%.

10. The apparatus of claim 1, wherein the protective material comprises an elastic modulus of at least about 2 to 8 kPa.

11. The apparatus of claim 1, wherein the protective material comprises an elastic modulus of at least about 4 to 6 kPa.

12. The apparatus of claim 1, wherein the protective material comprises an elastic modulus of at least about 4.5 to 5.5 kPa.

13. The apparatus of claim 1, wherein the protective material comprises a pore size of at least about 15 to 20 μm.

14. The apparatus of claim 1, wherein the nonabsorbable material comprises a synthetic polymer.

15. The apparatus of claim 1, wherein the nonabsorbable material comprises a synthetic polymer and the synthetic polymer comprises polyurethane.

16. The apparatus of claim 1, wherein the protective material comprises silicone.

17. The apparatus of claim 1, wherein the porous bioabsorbable cryogel is self-assembled.

18. The apparatus of claim 1, wherein the protective material is positioned adjacent to the nonabsorbable material.

19. The apparatus of claim 1, wherein the protective material is located at a selected surface of the nonabsorbable material.

20. The apparatus of claim 1, wherein the protective material is in contact with the nonabsorbable material such that a portion of the protective material is interspersed within the nonabsorbable material.

21. The apparatus of claim 1, wherein the nonabsorbable material comprises fenestrations.

22. The apparatus of claim 1, comprising a source of suction in gaseous communication with the nonabsorbable material and the protective material.

23. A method for preparing a tissue repair material comprising a tissue protective cryogel and a nonabsorbable foam, the method comprising the steps of:
a. forming a solution of hyaluronic acid and silk fibroin; and
b. applying the solution of hyaluronic acid and silk fibroin as the tissue protective cryogel to the nonabsorbable foam to obtain the tissue repair material.

24. The method of claim 23, comprising the step of preparing silk fibroin by extracting and purifying silk fibroin from raw silk.

25. The method of claim 23, wherein the hyaluronic acid concentration is at least about 30 to 40% by weight.

26. The method of claim 23, wherein the step of applying the solution of hyaluronic acid and silk fibroin as the tissue protective cryogel comprises lyophilizing the solution of hyaluronic acid and silk fibroin onto a surface of the nonabsorbable foam.

27. The method of claim 23, wherein the step of applying the solution of hyaluronic acid and silk fibroin as the tissue protective cryogel comprises injecting the tissue protective cryogel at a surface of the nonabsorbable foam.

28. The method of claim 23, comprising the step of sonicating the solution of hyaluronic acid and the silk fibroin with a sonicator.

29. The method of claim 23, comprising the step of vortexing the solution of hyaluronic acid and the silk fibroin with a vortexer.
